(19) United States
(12) Patent Application Publication
(10) Pub. No.: US 2020/0344227 A1
(43) Pub. Date: Oct. 29, 2020

(54) TECHNIQUES FOR VALIDATING USER CORRELATION TO SENSOR DATA

(71) Applicant: PHILIPS HEALTHCARE INFORMATICS INC., Forster City, CA (US)

(72) Inventor: SOUMYA DAS, San Diego, CA (US)

(21) Appl. No.: 16/764,911

(22) PCT Filed: Oct. 19, 2018
(86) PCT No.: PCT/US2018/056647

§ 371 Date: May 18, 2020

(51) Int. Cl. H04L 29/06; H04W 12/06; G06Q 10/10; G06Q 50/26; G06Q 30/00; G06F 7/58; G06K 9/00; A61B 5/1172; A61B 5/1171; A61B 5/0402; A61B 5/0205; A61B 5/11

(52) U.S. Cl. CPC H04L 63/0861; G16H 20/30; H04L 63/083; G06Q 10/10; G06Q 50/265; G06Q 30/0185; G06F 7/58; G06K 9/00892; A61B 5/1172; A61B 5/1176; A61B 5/0402; A61B 5/0205; A61B 5/112; G06K 9/00617; H04W 12/06

(57) ABSTRACT

Disclosed is a method and apparatus for validating user correlation to sensor data. The method for determining whether sensor data corresponds to a user may include obtaining one or more sensor data sets, wherein each sensor data set is associated with one or more validation indicators. The method may include identifying at least one sensor data set from the one or more sensor data sets based on the one or more validation indicators of each sensor data set. Additionally, the method may include determining whether the identified at least one sensor data set corresponds to a user based on a comparison of sensor data from the identified at least one sensor data set to a user sensor data template. Furthermore, the method may include reporting the determination of whether the identified at least one sensor data set corresponds to the user.

### CLAIM OF PRIORITY UNDER 35 U.S.C. § 119

The present Application for Patent claims priority to Non-Provisional Application No. 15/814,787 entitled “TECHNIQUES FOR VALIDATING USER CORRELATION TO SENSOR DATA” filed Nov. 16, 2017, assigned to the assignee hereof and hereby expressly incorporated by reference herein.

### FIELD

This disclosure relates generally to techniques for data validation/authentication and more particularly to techniques, methods and apparatuses for validating user correlation to sensor data.

### BACKGROUND

Currently, insurance companies are starting to provide wellness programs that incentivizes participants by providing money for the participants to exercise a certain amount each day, week, month, etc. This allows the insurance companies to promote healthier participants while giving the participant a positive reward for this health behavior. This process requires a user to wear a fitness band or fitness device/tracker that is associated with insurance company and the insurance company receives the exercise data such as step count and credits the user's account accordingly.

However, the insurance company and/or the wellness plan operator is taking it on good faith that the participant/user is in fact the one who performs the exercise associated with the step count collected and reported, which may not be true. For example, a participant may provide their fitness tracker to a third party (such as spouse that exercises regularly) and have the third party perform the exercise needed for the step count on the participant's behalf, which runs counter to the purpose of the program.

### SUMMARY

An example of a method for determining whether sensor data corresponds to a user may include obtaining one or more sensor data sets, wherein each sensor data set is associated with one or more validation indicators. The method may include identifying at least one sensor data set from the one or more sensor data sets based on the one or more validation indicators of each sensor data set. Additionally, the method may include determining whether the identified at least one sensor data set corresponds to a user based on a comparison of sensor data from the identified at least one sensor data set to a user sensor data template. Furthermore, the method may include reporting the determination of whether the identified at least one sensor data set corresponds to the user.

An example of a device for determining whether sensor data corresponds to a user, the device may comprise one or more transceivers, a memory and one or more processors coupled to the memory and the one or more transceivers. The one or more processors configured to obtain one or more sensor data sets, wherein each sensor data set is associated with one or more validation indicators. The one or more processor may be configured to identify at least one sensor data set from the one or more sensor data sets based on the one or more validation indicators of each sensor data set. Additionally, the one or more processor may be configured to determine whether the identified at least one sensor data set corresponds to a user based on a comparison of sensor data from the identified at least one sensor data set to a user sensor data template. Furthermore, the device may include one or more processor may be configured to report the determination of whether the identified at least one sensor data set corresponds to the user.

An example of a device for determining whether sensor data corresponds to a user may include obtaining one or more sensor data sets, wherein each sensor data set is associated with one or more validation indicators. The device may include means for identifying at least one sensor data set from the one or more sensor data sets based on the one or more validation indicators of each sensor data set. Additionally, the device may include means for determining whether the identified at least one sensor data set corresponds to a user based on a comparison of sensor data from the identified at least one sensor data set to a user sensor data template. Furthermore, the device may include means for reporting the determination of whether the identified at least one sensor data set corresponds to the user.

An example of a non-transitory computer-readable medium for determining whether sensor data corresponds to a user comprising processor-executable program code configured to cause a processor to obtain one or more sensor data sets, wherein each sensor data set is associated with one or more validation indicators. The processor-readable instructions configured to cause a processor to identify at least one sensor data set from the one or more sensor data sets based on the one or more validation indicators of each sensor data set. Additionally, the processor-readable instructions configured to cause a processor to determine whether the identified at least one sensor data set corresponds to a user based on a comparison of sensor data from the identified at least one sensor data set to a user sensor data template. Furthermore, the processor-readable instructions configured to cause a processor to report the determination of whether the identified at least one sensor data set corresponds to the user.

### BRIEF DESCRIPTION OF THE DRAWINGS

Non-limiting and non-exhaustive aspects are described with reference to the following figures, wherein like reference numerals refer to like parts throughout the various figures unless otherwise specified.

FIG. 1 shows an example of a communication environment in which various aspects of the disclosure may be implemented.

FIG. 2 shows a flow diagram illustrating an example method of validating user correlation to sensor data, according to some aspects of the disclosure.

FIG. 3 is block diagram of an example mobile device and the components within the mobile device in which aspects of the disclosure may be implemented

FIG. 4 is block diagram of an example server and the components within the mobile device in which aspects of the disclosure may be implemented

### DETAILED DESCRIPTION

References throughout this specification to one implementation, an implementation, an embodiment, an embodiment, and/or the like mean that a particular feature, structure, characteristic, and/or the like described in relation to a particular implementation and/or embodiment is included in at least one implementation and/or embodiment of claimed subject matter. Thus, appearances of such phrases, for example, in various places throughout this specification are not necessarily intended to refer to the same implementation and/or embodiment or to any one particular implementation and/or embodiment. Furthermore, it is to be understood that particular features, structures, characteristics, and/or the like described are capable of being combined in various ways in one or more implementations and/or embodiments and, therefore, are within intended claim scope. However, these and other issues have a potential to vary in a particular context of usage. In other words, throughout the disclosure, particular context of description and/or usage provides helpful guidance regarding reasonable inferences to be drawn; however, likewise, “in this context” in general without further qualification refers to the context of the present disclosure.

The features and advantages of the disclosed method and apparatus will become more apparent to those skilled in the art after considering the following detailed description in connection with the accompanying drawing.

Systems and techniques herein provide for validating user correlation to sensor data.

Some solutions may include validating a portion of the sensor data at a particular time; however, in situations where validation occurs at a static time period, such as the beginning of an exercise, it allows the user to identify these time periods and try to “cheat” the system by validating during these time periods and handing it off to a third party to complete the task thus bypassing the intended effect of the wellness bands which are to make the user more active and thus healthier.

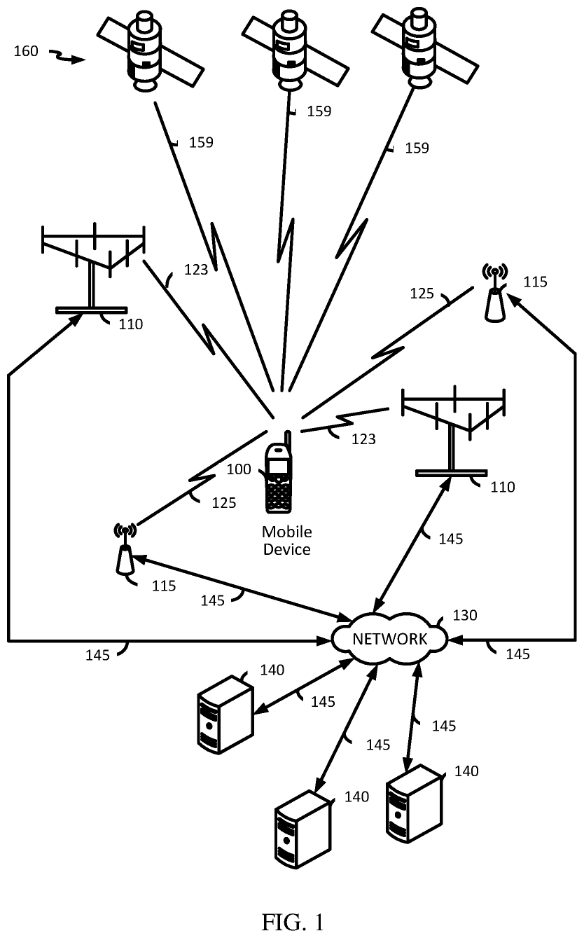

FIG. 1 shows an example of a communication environment. The mobile device 100 may transmit radio signals to, and receive radio signals from, a wireless communication network. In one example, mobile device 100 may communicate with a cellular communication network by transmitting wireless signals to, or receiving wireless signals from one or more cellular transceivers 110 which may comprise a wireless base transceiver subsystem (BTS), eNodeB transceiver or an evolved NodeB (eNodeB) transceiver over wireless communication links 123. Similarly, mobile device 100 may transmit wireless signals to, or receive wireless signals from local transceiver 115 over wireless communication link 125. A local transceiver 115 may comprise an access point (AP), femtocell, Home Base Station, small cell base station, Home Node B (HNB) or Home eNodeB (HeNB) and may provide access to a wireless local area network (WLAN, e.g., IEEE 802.11 network), a wireless personal area network (WPAN, e.g., Bluetooth® network) or a cellular network (e.g. an LTE network or other wireless wide area network such as those discussed in the next paragraph). These are merely examples of networks that may communicate with a mobile device 100 over a wireless link, and claimed subject matter is not limited in this respect.

Additionally, the mobile device 100 may communicate with the server 140 through a communication network (e.g. communication link 125 and wireless communication link 123), as described above, or it may also and/or alternatively communicate via a peer-to-peer or a mesh network (e.g. wireless communication link 150).

The mobile device 100 may be a mobile device such as a smartphone, a tablet computer, a personal computer, a laptop or netbook, a smart watch, a head-mounted display (HMD), other wearable device, diagnostic device, or a headless (i.e. without a display) device.

The mobile device 100 may be a fixed device. For example, the mobile device 100 may be an access point/base station or collocated with the access point/base station, a device that is attached to an access point/base station, a dedicated device, etc.

Additionally, the mobile device 100 may be part of a larger device such as a router, airplane, vehicle, smart appliances, etc. In an example, the mobile device 100 may be collocated or embedded in a vehicle.

Examples of network technologies that may support wireless communication link 123 are Global System for Mobile Communications (GSM), Code Division Multiple Access (CDMA), Wideband CDMA (WCDMA), Long Term Evolution LTE), High Rate Packet Data (HRPD), 5G. GSM, WCDMA and LTE are technologies defined by 3GPP. CDMA and HRPD are technologies defined by the 3rd Generation Partnership Project 2 (3GPP2). WCDMA is also part of the Universal Mobile Telecommunications System (UMTS) and may be supported by an HNB. Cellular transceivers 110 may comprise deployments of equipment providing subscriber access to a wireless telecommunication network for a service (e.g., under a service contract). Here, a cellular transceiver 110 may perform functions of a cellular base station in servicing subscriber devices within a cell determined based, at least in part, on a range at which the cellular transceiver 110 is capable of providing access service. Examples of radio technologies that may support wireless communication link 125 are IEEE 802.11, Bluetooth® (BT) and LTE.

In a particular implementation, cellular transceivers 110 and local transceiver 115 may communicate with servers 140 over a network 130 through links 145 or may communicate directly with servers 140 (not shown) via a peer-to-peer connection or mesh network. Here, network 130 may comprise any combination of wired or wireless links and may include cellular transceiver 110 and/or local transceiver 115 and/or servers 140. In a particular implementation, network 130 may comprise Internet Protocol (IP) or other infrastructure capable of facilitating communication between mobile device 100 and servers 140 through local transceiver 115 or cellular transceiver 110. In an embodiment, network 130 may also facilitate communication between mobile device 100, servers 140. In another implementation, network 130 may comprise cellular communication network infrastructure such as, for example, a base station controller or packet based or circuit based switching center (not shown) to facilitate mobile cellular communication with mobile device 100. In a particular implementation, network 130 may comprise local area network (LAN) elements such as WLAN APs, routers and bridges and may in that case include or have links to mobile device elements that provide access to wide area networks such as the Internet. In other implementations, network 130 may comprise a LAN and may or may not have access to a wide area network but may not provide any such access (if supported) to mobile device 100. In some implementations network 130 may comprise multiple networks (e.g., one or more wireless networks and/or the Internet). In one implementation, network 130 may include one or more serving mobile device or Packet Data Network mobile device.

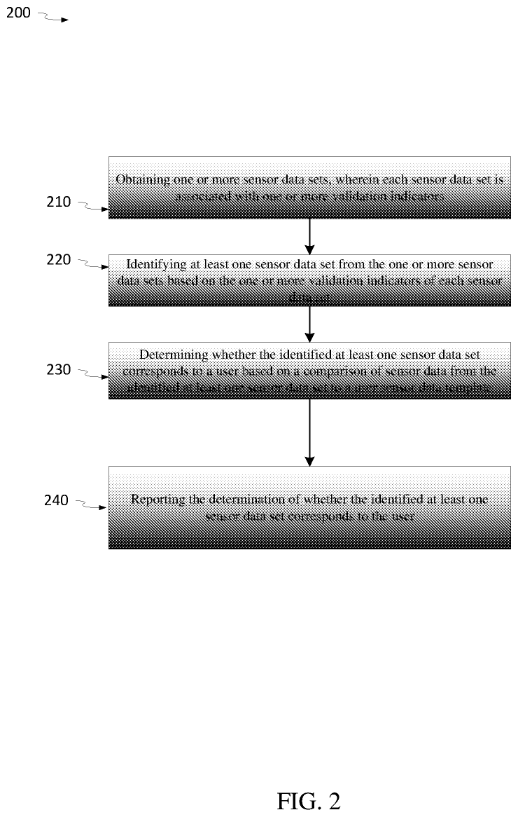

FIG. 2 shows a process diagram for determining whether the sensor data corresponds to a user. At block 210, the device 100 obtains one or more sensor data sets. This may be obtained from one or more sensors, such as an accelerometer, gyroscope, visual sensor, fingerprint sensor, etc.

The sensor data set may include one or more sensor measurements from one or more sensors. In an embodiment, the sensor data set may also include a timestamp of when the one or more sensor measurements were taken. The timestamp may be a timestamp for each sensor measurement, a starting timestamp and ending timestamp, or a starting timestamp/ending timestamp and a duration.

In an implementation, the sensor data may also include which sensor(s) provided the sensor measurement(s). For example, if the sensor measurements are grouped so that it includes sensor data from each sensor for a time period, the sensor data may indicate which sensor corresponds to which measurement. For example, the first in the group is a measurement from an accelerometer and the second in the group is a measurement from a gyroscope.

The sensor data may be captured and/or obtained using the same goals as described in the specification and/or it may be different goals. For example, the sensor data may be captured based on number of steps per minutes or number of consecutive activity with an allowance of a few seconds between step whereas the user's activity goal may be based on number of steps per activity or number of steps per day.

The sensor data set is associated with one or more validation indicators. The one or more validation indicators may be a random number or a pseudo random number. For example, the one or more validation indicators may be based a pseudo random number based on the sensor data set timestamp. The validation indicator provides the benefit of randomizing or validating data that may be impactful toward the user's goals so it is less likely that a user can “cheat” the system by having someone else complete the goals for them.

In an implementation, the one or more validation indicators may be based on the impact of the sensor data set on one or more user goals. For example, if the mobile device 100 is used on a run for five miles and the user's goal is to have steps that are equivalent to ten miles then the one or more validation indicators may indicate the sensor data set has a large impact on the user's goals and may even provide an approximate or exact number relating to the impact on the user's goals. In an implementation, the impact may be predefined values or based on the user's goals, such as a percentage of the user's goals.

The impact may also be an estimate based on the user's historical pattern. For example, if the user always runs at a certain time of day, such as six am every day, the impact may be estimated based on the user's typical exercise and the impact it has on the user's goals.

The one or more validation indicators may be based on the device's location when the sensor data set was generated or obtained. For example, if the device is located at a national park close or on a hiking trail then the validation indicator may be high, because it may indicate that the user is likely going to be performing exercise for a long duration. In another example, if the device location is at a major league baseball stadium then the validation indicator may be lower because it unlikely that the user is performing exercise but instead watching a baseball game.

In some circumstances, the user may be “cheating” the system by having someone else perform a number of small exercises throughout the day that are below a threshold (e.g. below a thousand steps) but the user performs one exercise that is more impactful and greater than the threshold of the small exercises (e.g. greater than a thousand steps), so they may be forcing the device to validate the impactful exercise but totality of the small exercises together may be more impactful (e.g. a person other than the user is performing nine exercises a day that are a thousand steps a piece). The device may identify a pattern and set a validation indicator based on the identified pattern.

According to an aspect of the disclosure, the one or more validation indicators may be based on how close the user is to meeting or exceeding the user's goals, whether the sensor data set will meet or exceed the user's goals or any combination thereof. For example, if the user is a hundred steps away from their goal of walking a thousand steps the validation indicator for upcoming sensor data sets may be higher because the likelihood that it will cause the user to complete their goal will be higher. Similarly, if the sensor data contains data that indicates the user has taken a hundred or more steps that causes the user to complete the goal then that may be associated with a higher validation indicator.

The association of the one or more validation indicators may be done while the device 100 is still obtaining sensor data sets. For example, after the first sensor measurement or after it meets a certain threshold, the device 100 may associate one or more validation indicators to the sensor data set that include the first sensor measurement and all future sensor measurements until a second threshold is met, such as a period of reduced or no activity. For example, if the user starts a run that may meet a first threshold so a validation indicator is associated with the current activity set includes all future sensor data until the user stops the activity for a particular amount of time, such as stopped running/walking for five minutes, this may satisfy the second threshold so all the sensor data obtained in the time period between when the first and second threshold were met will be associated with the determined validation indicator.

According to an aspect of the disclosure, the validation indicators may be determined and associated after a complete sensor data set has been obtained. The validation indicators may include various values such as impact, variability, uncertainty, etc. The impact value that is part of the validation indicators may be determined based on the complete sensor data set impact on the user's overall goals or relative to the rest of the obtained sensor data set.

The variability may be determined based on a comparison of the current complete sensor data set, a comparison of the current complete sensor data set to previously obtained sensor data sets, or any combination thereof. For example, if a sensor data set includes some running and lots of walking then the validation indicators may include a variability value that indicates high variability in the sensor data set, and this may indicate that this sensor data set may have a low need to be validated unless if it is also associated with a high impact value.

The uncertainty may be determined based on changes from historical patterns. For example, if the user is typically sedentary for during working hours on weekdays but on another day during working hours the sensor data indicates the user is being very active then the uncertainty value may increase so it can increase the likelihood of the need for validation.

In an implementation, the device may change how it determines the validation indicators based on the day, time, number of potential violation, number of previous violations, third party input, random selection, or any combination thereof.

In an example, the third party, such as an insurance provider, may identify patterns that may cause the insurance provider to want additional validation for the user, so they may request that the device determine the validation indicators in one or more of the ways specified in this disclosure.

In cases where the sensor data sets indicate potential violations or has previously indicating violations (e.g. the sensor data set does not correspond to the user) the way the validation indicators may be determined using a different technique as specified in the disclosure, so the user is unable to figure out how the device is validating the sensor data set.

In one implementation, if the number of violations (e.g. where the sensor data set does not correspond to the user) meets or exceeds one or more thresholds then the user may be flagged on the device and/or on the one or more servers. In one example, after the number of violations meets a first threshold and this constitutes the users first violation then a warning or notification may be provided to the user, on the user's second reported violation it may remove part of the rewards, such as reducing the reward pool or returning rewards that have already been received or accumulated. As provided in the example, there may be a tiered approach to dealing with the violations.

Additionally, the violations may be reported to the user but there may be a threshold for number of reported violations before the one or more servers, i.e. the service provider, is notified of the violations.

At block 220, at least one sensor data set is identified from the one or more sensor data sets based on the one or more validation indicators. In one implementation, the one or more validation indicators with the highest values may be used to identify the corresponding at least one sensor data set. In another implementation, the one sensor data set may be identified based on the validation indicators that indicate high impact.

According to some aspects of the disclosure, if there are more than one sensor data set identified based on similar validation indicators then at least one of identified sensor data set may be provided for comparison, as described later in the specification. It may narrow the identified sensor data set by randomly or pseudo-randomly selecting at least one of the sensor data sets, by selecting at least one sensor data set based on the sensor data set timestamp, by selecting at least one sensor data set based on relative position in a queue compared to the queue position of the rest of the identified sensor data sets, or any combination thereof.

The identification of at least one sensor data set may occur at a particular time of day, after the validation indicators reach a particular threshold, the user's goals, if the sensor data set meets one or more thresholds, on request from a third party or user, or any combination thereof.

In an example, the device may accumulate sensor data sets that each correspond with one or more validation indicators, but rather than validate each and every sensor data set it may wait until five pm every day and identify which sensor data set to validate based on the validation indicators.

As another example, the device may accumulate one or more sensor data sets but if any of those data sets are associated with one or more validation indicators that meet or exceed a threshold then the corresponding sensor data set(s) may be validated. This may similarly be done using a comparison of the sensor data and one or more threshold. For example, if the sensor data exceeds a thousand steps and the threshold is a thousand sets then that sensor data may be validated.

The user's goals may also be used to determine when to identify the at least one sensor data set. For example, if the user hasn't met their goals for the day then the device may periodically or pseudo randomly accumulate sensor data sets and identify at least one sensor data set to validate; however, if the user's goals have already been met then it may accumulate sensor data sets until midnight and identify sensor data sets to validate at that time.

A third party or the user may also request when sensor data should be validated and this may trigger the identification of the at least one sensor data set from the accumulated sensor data sets.

At block 230, a device determines whether the identified at least one sensor data set corresponds to a user based on a comparison of the identified at least one sensor data set and a user sensor data template.

The user sensor data template may be determined from user sensor data enrollment (e.g. user's initial calibration), from another device's enrollment (e.g. gait analysis at a doctor's office), or any combination thereof. For example, when the user initial setups the device it may ask the user to perform one or more steps for the enrollment of the device, such as a casual walk, a jog, run, etc. In another example, the device may require the user to hold the camera up to the user's face so pictures of the user's face can be captured at different points in the enrollment process to authenticate that the user is one enrolling the device and not another user. In another example, the user sensor data template may be obtained in a controlled environment, such as on a treadmill at a doctor's office, where the user is asked to walk for a certain duration and/or distance.

The sensor data from the identified sensor data set may be used to compare the similarity to the user sensor data template. This comparison may be based on the characteristics of sensor data (i.e. accelerometer data) to the characteristics of user sensor data template. For example, one characteristic may be the distance between two peaks in accelerometer data to indicate the user's gait and that distance may be compared to the distance between peaks of accelerometer data in the user sensor data template. It may use a wide variety of characteristics, such as: distance between peaks, time from peak to trough, distance between troughs, amplitude of peaks/troughs, pulse shapes, integration of the sensor data or any combination thereof.

According to an aspect of the disclosure, the user sensor data template may be one or more thresholds. If the identified sensor data set meets or exceeds the one or more thresholds then the device may indicate that the sensor data sets correspond to the user.

In an implementation, if the identified sensor data does not meet or exceed the one or more thresholds or is not similar to the user sensor data template, the device may request a user's passcode, user's biometric data or any combination thereof. For example, if a user is running but changing their running form but the comparison is based on gait data then the user sensor data template may provide a different gait from the user's new running form so it may indicate the sensor data is not the user.

Rather than indicating it is not the user, it may provide a fallback option for the user to provide their passcode and/or biometric information to authenticate that it is in fact the user. The biometric information may be the user's pulse(s), heart rate, blood pressure, facial data, iris data, fingerprint data, gait data, electrocardiogram or any combination thereof. As an example, the user's iris or facial features may be used to authenticate the user and to validate that the sensor data sets correspond to the user. In another example, one or more characteristics of the user's pulse(s) may be used to authenticate the user, such as using the pulse shape to identify whether or not it corresponds to the user based on a comparison of the pule shape to the user's security data that contains the user's pulse shape.

In the case of additional gait analysis, the device may indicate to the user that it can't authenticate the user and it may ask the user to perform a task, such as a walk, with the user's normal form and if the user is able to perform the task that is acceptably similar to the user sensor data template then it can move out of the fallback mode. In one implementation, once the device comes out of the fallback mode it may perform a new enrollment procedure. In another implementation, it may identify the number of times or the frequency of fallback incidents before the device triggers a new enrollment procedure.

The passcode and/or biometric information may be compared against the user's security data and if it matches or in the case of the biometric information is similar then it would indicate that the sensor data sets correspond to the user.

At block 240, a device may report the determination of whether the identified at least one sensor data set corresponds to the user. For example, the one or more sensor data sets may be determined to correspond to the user, so the device may report this information to a third party or the user. As an example, an insurance provider may be incentivizing the user to exercise by providing monetary credits, etc. so if the sensor data did not correspond to the user then the insurance company may be notified so the incentives are not provided to the user.

In another example, a device may report that the one or more sensor data sets do not correspond to the user in response to the determination that the identified at least one sensor data set do not corresponds to the user. The device may report this information to a third party or the user. As an example, an insurance provider may be incentivizing the user to exercise by providing monetary credits, etc. so if the sensor data did not correspond to the user then the insurance company may be notified so the incentives are not provided to the user.

The processes described in FIG. 2 and/or throughout the specification may be performed by a mobile device, such as a wearable device and/or smartphone, an access point, such as a gateway and/or a smartphone, a service platform, such as a server, or any combination thereof.

The processes described in FIG. 2 and/or throughout the specification provide various benefits. For example, it discourages a user from utilizing a third party to perform exercise for them, because they are unable to determine which sensor data set will be validated against the user's signature because the sensor data set may be validated in a non-deterministic manner.

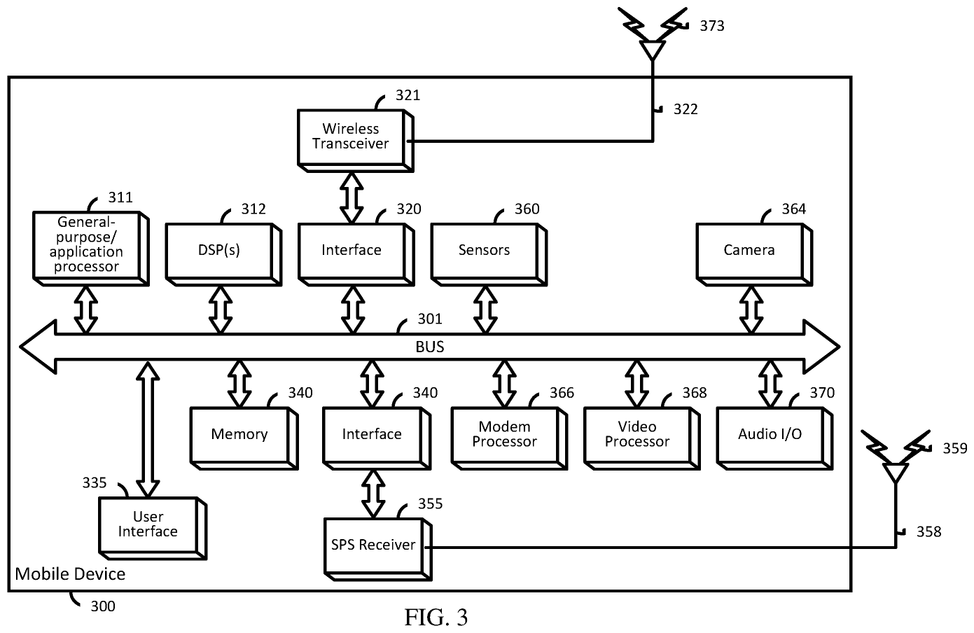

FIG. 3 is a schematic diagram of mobile device 300 in which various aspects of the disclosure may be implemented. Mobile device 100 shown in FIG. 1 may comprise one or more features of mobile device 300 shown in FIG. 3. In certain implementations, mobile device 300 may comprise a wireless transceiver 321 which is capable of transmitting and receiving wireless signals 323 via wireless antenna 322 over a wireless communication network. Wireless transceiver 321 may be connected to bus 301 by a wireless transceiver bus interface 320. Wireless transceiver bus interface 320 may, in some implementations be at least partially integrated with wireless transceiver 321. Some implementations may include multiple wireless transceivers 321 and wireless antennas 322 to enable transmitting and/or receiving signals according to corresponding multiple wireless communication standards such as, for example, versions of IEEE Standard 802.11, CDMA, WCDMA, LTE, UMTS, GSM, AMPS, Zigbee, Bluetooth and a 5G or NR radio interface defined by 3GPP, just to name a few examples. In a particular implementation, wireless transceiver 321 may transmit signals on an uplink channel and receive signals on a downlink channel as discussed above.

Mobile device 300 may also comprise SPS receiver 355 capable of receiving and acquiring SPS signals 359 via SPS antenna 558 (which may be integrated with antenna 522 in some implementations). SPS receiver 355 may also process, in whole or in part, acquired SPS signals 359 for estimating a location of mobile device 300. In some implementations, general-purpose processor(s) 311, memory 340, digital signal processor(s) (DSP(s)) 312 and/or specialized processors (not shown) may also be utilized to process acquired SPS signals, in whole or in part, and/or calculate an estimated location of mobile device 300, in conjunction with SPS receiver 355. Storage of SPS or other signals (e.g., signals acquired from wireless transceiver 321) or storage of measurements of these signals for use in performing positioning operations may be performed in memory 340 or registers (not shown). General-purpose processor(s) 311, memory 340, DSP(s) 312 and/or specialized processors may provide or support a location engine for use in processing measurements to estimate a location of mobile device 300. In a particular implementation, all or portions of actions or operations set forth for process 300 may be executed by general-purpose processor(s) 311 or DSP(s) 312 based on machine-readable instructions stored in memory 340.

Also shown in FIG. 3, digital signal processor(s) (DSP(s)) 312 and general-purpose processor(s) 311 may be connected to memory 340 through bus 301. A particular bus interface (not shown) may be integrated with the DSP(s) 312, general-purpose processor(s) 311 and memory 340. In various implementations, functions may be performed in response to execution of one or more machine-readable instructions stored in memory 340 such as on a computer-readable storage medium, such as RAM, ROM, FLASH, or disc drive, just to name a few examples. The one or more instructions may be executable by general-purpose processor(s) 311, specialized processors, or DSP(s) 312. Memory 340 may comprise a non-transitory processor-readable memory and/or a computer-readable memory that stores software code (programming code, instructions, etc.) that are executable by processor(s) 311 and/or DSP(s) 312. The processor(s) 311 and/or the DSP(s) 312 in conjunction with memory 340 may be used to perform various operations as described throughout the specification. For example, the processor(s) 311 and/or the DSP(s) 312 in conjunction with memory 340 may be used to obtain one or more sensor data sets and each sensor data set may be associated with one or more validation indicators, similar to block 210. The processor(s) 311 and/or the DSP(s) 312 in conjunction with memory 340 may then identify at least one sensor data set from the one or more sensor data sets based on the one or more validation indicators, similar to block 220, and then determines whether the identified at least one sensor data set corresponds to a user based on a comparison of the identified at least one sensor data set and a user sensor data template, similar to block 230. In response to the identified at least one sensor data set corresponding to the user, then the processor 311 and/or the DSP(s) 312 in conjunction with the memory 340 may report that the one or more sensor data sets correspond to the user, similar to block 240. In response to the determination that the identified at least one sensor data set does not correspond to the user, then the processor 311 and/or the DSP(s) 312 in conjunction with the memory 340 may report that the one or more sensor data sets does not correspond to the user, similar to block 250. This may be reported to the device's display, not shown in the figure, and/or it may be provided via a wireless transceiver 321 to a remote device, such as a server. If the sensor data set does not correspond to the user, this may also be reported by the processor 311 and/or DSP(s) 312 in conjunction with the memory 340, similar to block 260. This may be reported to the device's display, not shown in the figure, and/or it may be provided via a wireless transceiver 321 to a remote device, such as a server.

Also shown in FIG. 3, a user interface 335 may comprise any one of several devices such as, for example, a speaker, microphone, display device, vibration device, keyboard, touch screen, just to name a few examples. In a particular implementation, user interface 335 may enable a user to interact with one or more applications hosted on mobile device 300. For example, devices of user interface 335 may store analog or digital signals on memory 340 to be further processed by DSP(s) 312 or general purpose processor 311 in response to action from a user. Similarly, applications hosted on mobile device 300 may store analog or digital signals on memory 340 to present an output signal to a user. In another implementation, mobile device 300 may optionally include a dedicated audio input/output (I/O) device 370 comprising, for example, a dedicated speaker, microphone, digital to analog circuitry, analog to digital circuitry, amplifiers and/or gain control. It should be understood, however, that this is merely an example of how an audio I/O may be implemented in a mobile device, and that claimed subject matter is not limited in this respect. In another implementation, mobile device 300 may comprise touch sensors 362 responsive to touching or pressure on a keyboard or touch screen device.

Mobile device 300 may also comprise a dedicated camera device 364 for capturing still or moving imagery. Camera device 364 may comprise, for example an imaging sensor (e.g., charge coupled device or CMOS imager), lens, analog to digital circuitry, frame buffers, just to name a few examples. In one implementation, additional processing, conditioning, encoding or compression of signals representing captured images may be performed at general purpose/application processor 311 or DSP(s) 312. Alternatively, a dedicated video processor 368 may perform conditioning, encoding, compression or manipulation of signals representing captured images. Additionally, video processor 368 may decode/decompress stored image data for presentation on a display device (not shown) on mobile device 300.

Mobile device 300 may also comprise sensors 360 coupled to bus 301 which may include, for example, inertial sensors and environment sensors. Inertial sensors of sensors 360 may comprise, for example accelerometers (e.g., collectively responding to acceleration of mobile device 300 in three dimensions), one or more gyroscopes or one or more magnetometers (e.g., to support one or more compass applications). Environment sensors of mobile device 300 may comprise, for example, temperature sensors, barometric pressure sensors, ambient light sensors, camera imagers, microphones, just to name few examples. Sensors 360 may generate analog or digital signals that may be stored in memory 340 and processed by DPS(s) 312 or general purpose application processor 311 in support of one or more applications such as, for example, applications directed to positioning or navigation operations. The user interface 335, sensors 360, camera(s) 364, touch sensors 362 or any combination thereof may be used to obtain the user's passcode and/or the user's biometric data such as the user's fingerprint, facial features, iris data, heart rate data, electrocardiogram, gait data, the user's pulses, etc.

In a particular implementation, mobile device 300 may comprise a dedicated modem processor 366 capable of performing baseband processing of signals received and down converted at wireless transceiver 321 or SPS receiver 355. Similarly, modem processor 366 may perform baseband processing of signals to be upconverted for transmission by wireless transceiver 321. In alternative implementations, instead of having a dedicated modem processor, baseband processing may be performed by a general-purpose processor or DSP (e.g., general purpose/application processor 311 or DSP(s) 312). It should be understood, however, that these are merely examples of structures that may perform baseband processing, and that claimed subject matter is not limited in this respect.

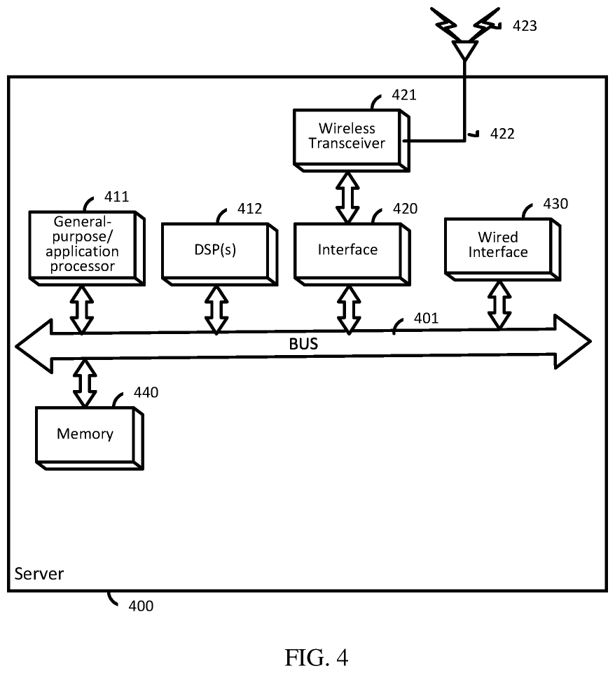

FIG. 4 is a schematic diagram of server 400 in which various aspects of the disclosure may be implemented. Server 140 shown in FIG. 1 may comprise one or more features of the server 400 shown in FIG. 4. In certain implementations, server 400 may comprise a wireless transceiver 421 which is capable of transmitting and receiving wireless signals 423 via wireless antenna 422 over a wireless communication network. Wireless transceiver 421 may be connected to bus 401 by a wireless transceiver bus interface 420. Wireless transceiver bus interface 420 may, in some implementations be at least partially integrated with wireless transceiver 421. Some implementations may include multiple wireless transceivers 421 and wireless antennas 422 to enable transmitting and/or receiving signals according to corresponding multiple wireless communication standards such as, for example, versions of IEEE Standard 802.11, CDMA, WCDMA, LTE, UMTS, GSM, AMPS, Zigbee, Bluetooth and a 5G or NR radio interface defined by 3GPP, just to name a few examples. In a particular implementation, wireless transceiver 421 may transmit signals on an uplink channel and receive signals on a downlink channel as discussed above.

The server 400 may include a wired interface 430, such as an ethernet, DSL, cable modem, etc. The wired interface 430 may be used use to send and/or receive data. This may be the one way the server is able to communicate with remote devices and/or may use the wireless transceiver bus interface 420, if available. The wireless transceiver bus interface 420 and/or the wired interface 430 of the server 400 may obtain one or more sensor data sets that are each associated with one or more validation indicators, similar to block 210. In one implementation, the mobile device 300 identifies at least one sensor data set from the one or more sensor data sets based on the one or more validation indicators of each sensor data set, similar block 220, but this information is sent from the mobile device 300 and received by the server 400 via the wired interface 430 and/or the wireless transceiver bus interface 420.

Also shown in FIG. 4, digital signal processor(s) (DSP(s)) 412 and general-purpose processor(s) 411 may be connected to memory 440 through bus 401. A particular bus interface (not shown) may be integrated with the DSP(s) 412, general-purpose processor(s) 411 and memory 440. In various implementations, functions may be performed in response to execution of one or more machine-readable instructions stored in memory 440 such as on a computer-readable storage medium, such as RAM, ROM, FLASH, or disc drive, just to name a few examples. The one or more instructions may be executable by general-purpose processor(s) 311, specialized processors, or DSP(s) 412. Memory 440 may comprise a non-transitory processor-readable memory and/or a computer-readable memory that stores software code (programming code, instructions, etc.) that are executable by processor(s) 411 and/or DSP(s) 412. The processor(s) 411 and/or the DSP(s) 412 in conjunction with memory 440 may be used to perform various operations as described throughout the specification. For example, the processor(s) 411 and/or the DSP(s) 412 in conjunction with memory 440 may be used to obtain one or more sensor data sets and each sensor data set may be associated with one or more validation indicators, similar to block 210. In one implementation, the server 400 may obtain the one or more sensor data with each sensor data set already associated with one or more validation indicators. The processor(s) 411 and/or the DSP(s) 412 in conjunction with memory 440 may then identify at least one sensor data set from the one or more sensor data sets based on the one or more validation indicators, similar to block 220. The processor(s) 411 and/or the DSP(s) 412 in conjunction with memory 440 may determine whether the identified at least one sensor data set corresponds to a user based on a comparison of the identified at least one sensor data set and a user sensor data template, similar to block 230. In response to the identified at least one sensor data set corresponding to the user, then the processor 411 and/or the DSP(s) 412 in conjunction with the memory 440 may report that the one or more sensor data sets correspond to the user, similar to block 240. In response to the determination that the identified at least one sensor data set does not correspond to the user, then the processor 411 and/or the DSP(s) 412 in conjunction with the memory 440 may report that the one or more sensor data sets do not correspond to the user, similar to block 250. This may be reported to the mobile device 300 and/or to a remote device, such as an insurance provider server, via a wireless transceiver 421 and/or the wired interface 430. If the sensor data set does not correspond to the user, this may also be reported by the processor 411 and/or DSP(s) 412 in conjunction with the memory 440, similar to block 260. This may be reported to the mobile device 300 and/or a remote device, such as an insurance provider server, via a wireless transceiver 421 and/or a wired interface 430.

Discussions of coupling between components in this specification do not require the components to be directly coupled. These components may be coupled directly or through one or more intermediaries. Additionally, coupling does not require they be directly attached, but it may also include electrically coupled, communicatively coupled or any combination thereof.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general-purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

Wireless communication techniques described herein may be in connection with various wireless communications networks such as a wireless wide area network (“WWAN”), a wireless local area network (“WLAN”), a wireless personal area network (WPAN), and so on. The term “network” and “system” may be used interchangeably herein. A WWAN may be a Code Division Multiple Access (“CDMA”) network, a Time Division Multiple Access (“TDMA”) network, a Frequency Division Multiple Access (“FDMA”) network, an Orthogonal Frequency Division Multiple Access (“OFDMA”) network, a Single-Carrier Frequency Division Multiple Access (“SC-FDMA”) network, 5G network, or any combination of the above networks, and so on. A CDMA network may implement one or more radio access technologies (“RATs”) such as cdma2000, Wideband-CDMA (“W-CDMA”), to name just a few radio technologies. Here, cdma2000 may include technologies implemented according to IS-95, IS-2000, and IS-856 standards. A TDMA network may implement Global System for Mobile Communications (“GSM”), Digital Advanced Mobile Phone System (“D-AMPS”), or some other RAT. GSM and W-CDMA are described in documents from a consortium named “3rd Generation Partnership Project” (“3GPP”). Cdma2000 is described in documents from a consortium named “3rd Generation Partnership Project 2” (“3GPP2”). 3GPP and 3GPP2 documents are publicly available. 4G Long Term Evolution (“LTE”) communications networks may also be implemented in accordance with claimed subject matter, in an aspect. A WLAN may comprise an IEEE 802.11x network, and a WPAN may comprise a Bluetooth network, an IEEE 802.15x, for example. Wireless communication implementations described herein may also be used in connection with any combination of WWAN, WLAN or WPAN.

In another aspect, as previously mentioned, a wireless transmitter or access point may comprise a cellular transceiver device, utilized to extend cellular telephone service into a business or home. In such an implementation, one or more mobile devices 100 may communicate with a cellular transceiver device via a code division multiple access (“CDMA”) cellular communication protocol, for example.

In the preceding detailed description, numerous specific details have been set forth to provide a thorough understanding of claimed subject matter. However, it will be understood by those skilled in the art that claimed subject matter may be practiced without these specific details. In other instances, methods and apparatuses that would be known by one of ordinary skill have not been described in detail so as not to obscure claimed subject matter.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein.

Therefore, it is intended that claimed subject matter not be limited to the particular examples disclosed, but that such claimed subject matter may also include all aspects falling within the scope of appended claims, and equivalents thereof.

For an implementation involving firmware and/or software, the methodologies may be implemented with modules (e.g., procedures, functions, and so on) that perform the functions described herein. Any machine-readable medium tangibly embodying instructions may be used in implementing the methodologies described herein. For example, software codes may be stored in a memory and executed by a processor unit. Memory may be implemented within the processor unit or external to the processor unit. As used herein the term “memory” refers to any type of long term, short term, volatile, nonvolatile, or other memory and is not to be limited to any particular type of memory or number of memories, or type of media upon which memory is stored.

If implemented in firmware and/or software, the functions may be stored as one or more instructions or code on a computer-readable storage medium. Examples include computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise RAM, ROM, EEPROM, CD-ROM or other optical disk storage, magnetic disk storage, semiconductor storage, or other storage devices, or any other medium that can be used to store desired program code in the form of instructions or data structures and that can be accessed by a computer; disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

In addition to storage on computer-readable storage medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims. That is, the communication apparatus includes transmission media with signals indicative of information to perform disclosed functions. At a first time, the transmission media included in the communication apparatus may include a first portion of the information to perform the disclosed functions, while at a second time the transmission media included in the communication apparatus may include a second portion of the information to perform the disclosed functions.

What is claimed is:

1. A method for determining whether sensor data corresponds to a user, the method comprising:
obtaining one or more sensor data sets, wherein each sensor data set is associated with one or more validation indicators;
identifying at least one sensor data set from the one or more sensor data sets based on the one or more validation indicators of each sensor data set;
determining whether the identified at least one sensor data set corresponds to a user based on a comparison of sensor data from the identified at least one sensor data set to a user sensor data template;
reporting the determination of whether the identified at least one sensor data corresponds to the user.

2. The method of claim 1, wherein the identifying the at least one sensor data set from the one or more sensor data sets is based on a comparison of the one or more validation indicators to a threshold, the at least one sensor data set with the highest one or more validation indicators, or any combination thereof.

3. The method of claim 1, further comprising:
in response to the one or more validation indicators exceeding a threshold, requesting a user's passcode, a user's biometric data or any combination thereof; and
wherein the determining whether the identified at least one sensor data set corresponds to a user is further based on a comparison of a user's passcode, a user's biometric data or any combination thereof.

4. The method of claim 1, wherein the one or more validation indicators is based on an impact of the sensor data on one or more user's goals, a random value, a pseudo random number, an instance after meeting one or more conditions, or any combination thereof.

5. The method of claim 3, wherein the user's biometric data comprises a user's fingerprint, facial features, iris data, heart rate, electrocardiogram, one or more user pulses, gait data, or any combination thereof.

6. The method of claim 1, wherein the reporting the determination of whether the identified at least one sensor data corresponds to the user comprises in response to a determination that the identified at least one sensor data does not correspond to the user, reporting that the identified at least one sensor data does not correspond to the user.

7. A device for determining whether sensor data corresponds to a user, the device comprising:
one or more transceivers;
a memory;
one or more processors coupled to the memory and the one or more transceivers, the one or more processors configured to:
obtain one or more sensor data sets, wherein each sensor data set is associated with one or more validation indicators;
identify at least one sensor data set from the one or more sensor data sets based on the one or more validation indicators of each sensor data set;
determine whether the identified at least one sensor data set corresponds to a user based on a comparison of sensor data from the identified at least one sensor data set to a user sensor data template;
report the determination of whether the identified at least one sensor data set corresponds to the user.

8. The device of claim 7, wherein the one or more processors are further configured to identify the at least one sensor data set from the one or more sensor data set is further based on a comparison of the one or more validation indicators to a threshold, the at least one sensor data set with the highest one or more validation indicators, or any combination thereof.

9. The device of claim 7, wherein the one or more processor is further configured to in response to the one or more validation indicators exceeding a threshold, request a user's passcode, a user's biometric data or any combination thereof; and wherein the one or more processors is further configured to determine whether the identified at least one sensor data set corresponds to a user is further based on a comparison of a user's passcode, a user's biometric data or any combination thereof.

10. The device of claim 7, wherein the one or more validation indicators is based on an impact of the sensor data on one or more user's goals, a random value, a pseudo random number, an instance after meeting one or more conditions, or any combination thereof.

11. The device of claim 9, wherein the user's biometric data comprises a user's fingerprint, facial features, iris data, heart rate, electrocardiogram, one or more user pulses or any combination thereof.

12. The device of claim 7, wherein the one or more processors is configured to report the determination of whether the identified at least one sensor data set corresponds to the user further comprises the one or more processors is configured to in response to a determination that the identified at least one sensor data does not correspond to the user, report that the identified at least one sensor data does not correspond to the user.

13. A device for determining whether sensor data corresponds to a user, the device comprising:
means for obtaining one or more sensor data sets, wherein each sensor data set is associated with one or more validation indicators;
means for identifying at least one sensor data set from the one or more sensor data sets based on the one or more validation indicators of each sensor data set;
means for determining whether the identified at least one sensor data set corresponds to a user based on a comparison of sensor data from the identified at least one sensor data set to a user sensor data template; and
means for reporting the determination of whether the identified at least one sensor data set corresponds to the user.

14. The device of claim 13, wherein the means for identifying the at least one sensor data set from the one or more sensor data set is further based on a comparison of the one or more validation indicators to a threshold, the at least one sensor data set with the highest one or more validation indicators, or any combination thereof.

15. The device of claim 13, further comprising:
in response to the one or more validation indicators exceeding a threshold, means for requesting a user's passcode, a user's biometric data or any combination thereof; and
wherein the means for determining whether the identified at least one sensor data set corresponds to a user is further based on a comparison of a user's passcode, a user's biometric data or any combination thereof.

16. The device of claim 13, wherein the one or more validation indicators is based on an impact of the sensor data on one or more user's goals, a random value, a pseudo random number, an instance after meeting one or more conditions, or any combination thereof.

17. The device of claim 15, wherein the user's biometric data comprises a user's fingerprint, facial features, iris data, heart rate, electrocardiogram, one or more user pulses or any combination thereof.

18. The device of claim 13, wherein the means for reporting the determination of whether the identified at least one sensor data set corresponds to the user comprises in response to a determination that the identified at least one sensor data does not correspond to the user, means for reporting that the identified at least one sensor data does not correspond to the user.

19. A non-transitory computer-readable medium for determining whether sensor data corresponds to a user comprising processor-executable program code configured to cause a processor to:
obtain one or more sensor data sets, wherein each sensor data set is associated with one or more validation indicators;
identify at least one sensor data set from the one or more sensor data sets based on the one or more validation indicators of each sensor data set;
determine whether the identified at least one sensor data set corresponds to a user based on a comparison of sensor data from the identified at least one sensor data set to a user sensor data template;
report the determination of whether the identified at least one sensor data set corresponds to the user.

20. The non-transitory computer-readable medium of claim 19, wherein the processor-executable program code is further configured to identify the at least one sensor data set from the one or more sensor data set is further based on a comparison of the one or more validation indicators to a threshold, the at least one sensor data set with the highest one or more validation indicators, or any combination thereof.

21. The non-transitory computer-readable medium of claim 19, wherein the processor-executable program code is further configured to in response to the one or more validation indicators exceeding a threshold, request a user's passcode, a user's biometric data or any combination thereof; and wherein the one or more processors is further configured to determine whether the identified at least one sensor data set corresponds to a user is further based on a comparison of a user's passcode, a user's biometric data or any combination thereof.

22. The non-transitory computer-readable medium of claim 19, wherein the one or more validation indicators is based on an impact of the sensor data on one or more user's goals, a random value, a pseudo random number, an instance after meeting one or more conditions, or any combination thereof.

23. The non-transitory computer-readable medium of claim 21, wherein the user's biometric data comprises a user's fingerprint, facial features, iris data, heart rate, electrocardiogram, one or more user pulses or any combination thereof.

24. The non-transitory computer-readable medium of claim 19, wherein the processor-executable program code is configured to report the determination of whether the identified at least one sensor data set corresponds to the user further comprises processor-executable program code is configured to in response to a determination that the identified at least one sensor data does not correspond to the user, report that the identified at least one sensor data does not correspond to the user.
